(19) United States
(12) Patent Application Publication
(10) Pub. No.: US 2009/0264788 A1
(43) Pub. Date: Oct. 22, 2009

(54) Nursing System of Visual Evoked Brain Wave Control Device

(76) Inventors: Kuo-kai SHYU, Jhongli City (TW); Po-lei Lee, Jhongli City (TW); Ming-huan Lee, Jhongli City (TW); Chi-hsun Wu, Jhongli City (TW); Jyun-jle Sie, Jhongli City (TW)

Correspondence Address: HDLS Patent & Trademark Services, P.O. BOX 220746, CHANTILLY, VA, 20153-0746, US

(21) Appl. No.: 12/179,660

(22) Filed: Jul. 25, 2008

(30) Foreign Application Priority Data
Apr. 16, 2008 (TW) 097113808

(51) Int. Cl. A61B 5/0476; A61G 7/015

(52) U.S. Cl. 600/544; 5/616

(57) ABSTRACT

A nursing system of a visual evoked brain wave control device, measuring a brain wave signal induced by the user when gazing at one optical display region, for controlling external devices is designed. The optical flash generating device receives an outer signal to flash the optical display region therein. A brain wave signal measurement device is used to measure a brain wave signal induced by users when gazing at the optical display region. A programmable chip is used to analyze the relation between the brain wave signal and the optical flash generating device, and control a selected option of the optical display region gazed by the users (for example, raising/falling of first, second or third portion of the hospital bed, volume adjustment of radio, channel adjustment, etc.), for improving the independence capability and the life quality of the users.

### BACKGROUND

1. Field of the Invention

The present invention relates to a nursing system of a visual evoked brain wave control device, and more specifically, to a nursing system controlled by a brain wave signal induced by a user gazing at least one optical display area of an optical flash generating device.

2. Description of the Related Art

With the rapid development of the mechanical and the electrical technologies, conventional manual hospital beds have been developed to become electrically adjustable ones. Some advanced hospital beds can be even controlled via user's voice commands. However, the aforementioned conventional hospital beds, which are adjusted by hand, by electrics or by voice, are not suitable for paralyzed users who are suffering from neural or muscular incapability (such as, quadriplegic patient, amyotrophic lateral sclerosis patient, or cerebellar atrophy patient, etc.). The patient still needs a nursing worker incessantly for adjusting the hospital beds.

Thus, the conventional hospital beds have following disadvantages.

Firstly, the patient cannot adjust the hospital beds by himself since the patient lacks movement capability.

Then, the patient needs the nursing worker incessantly, thus the nursing worker has a heavy burden.

Furthermore, the conventional hospital beds are not integrated with a periphery nursing device (such as, volume adjustment of radio, channel adjustment of radio, channel adjustment of TV, volume adjustment of TV, temperature adjustment, humidity adjustment, etc.). Thus the conventional hospital beds are inconvenient for the users.

What is needed is providing a nursing system, which can solve the above problems.

### BRIEF SUMMARY

A nursing system of a visual evoked brain wave control device in accordance with an exemplary embodiment of the present invention is provided. The nursing system is controlled by brain wave signals. The nursing system includes an optical flash generating device, a brain wave measurement device, a programmable chip and at least one controlled device. The optical flash generating device faces toward a user and is spaced a predetermined distance from eyes of the user such that the user directly gazes at least one optical display region of the optical flash generating device. The optical flash generating device is configured for receiving an outer signal to flash at least one optical display region. The brain wave measurement device is configured for measuring a brain wave signal induced by the user when gazing at one optical display region. The brain wave measurement device includes a brain measurement device, a biomedical signal instrument amplifier and an analog-to-digital converter arranged therein. The brain measurement device is configured for measuring the brain wave signal. The biomedical signal instrument amplifier is configured for amplifying the brain wave signal measured by the brain measurement device. The analog-to-digital converter is configured for converting the amplified brain wave signal to a digital signal. The programmable chip is configured for receiving the brain wave sent out from the brain wave measurement device, analyzing the relation between the brain wave and the optical flash generating device, and determining the selected option of the optical display region gazed by the user. Then, at least one controlled device is configured for receiving a signal sent out from the programmable chip, according to the selected option of the optical display region gazed by the user.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a schematic, frame diagram of a nursing system of a visual evoked brain wave control device, in accordance with an exemplary embodiment of the present invention;

FIG. 2 is a schematic, frame diagram of an optical flash generating device of the nursing system in FIG. 1; and

FIG. 3 is a schematic, application view of the nursing system in FIG. 1.

### DETAILED DESCRIPTION

Reference will now be made to the drawings to describe exemplary embodiments of the present nursing system of the visual evoked brain wave control device, in detail. The following description is given by way of example, and not limitation.

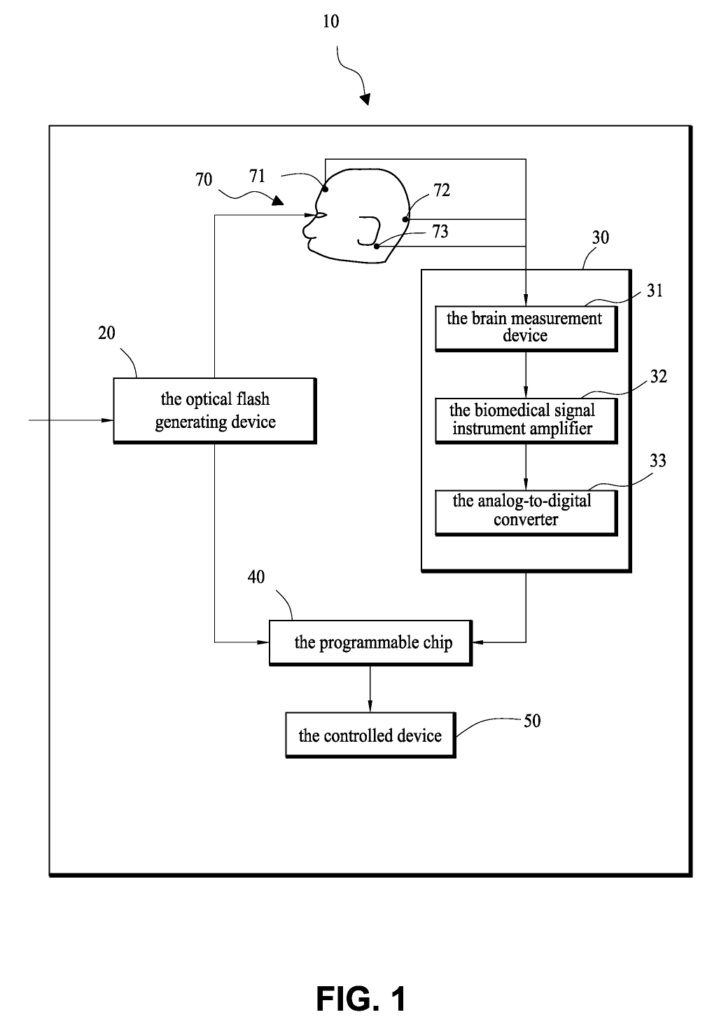

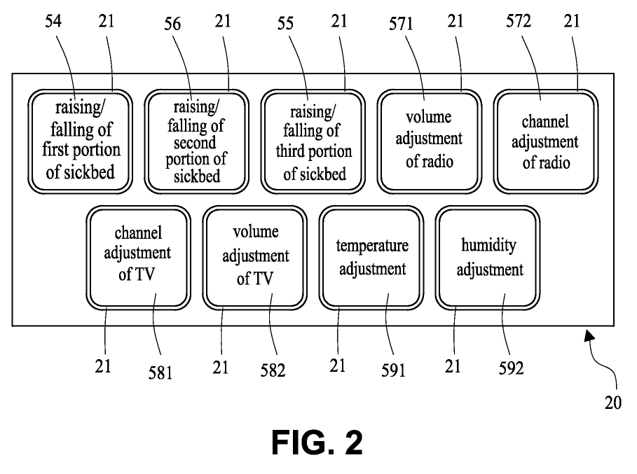

Referring to FIG. 1, a nursing system of a visual evoked brain wave control device, in accordance with an exemplary embodiment of the present invention, is provided. The nursing system 10 includes an optical flash generating device 20, a brain wave signal measurement device 30, a programmable chip 40 and at least one controlled device 50. The optical flash generating device 20 faces toward a user and is spaced a predetermined distance (e.g. 30 to 60 centimeters) from eyes of the user, such that the user may directly view at least one optical display area 21 of the optical flash generating device 20. The optical flash generating device 20 receives an outer signal to drive the at least one optical display area 21 such that the at least one optical display area 21 flashes respectively. Then, at least one optical display area 21 corresponds to at least one controlled device 50. The controlled device 50 (as shown in FIG. 2) may be one of raising/falling 54 of a first portion of a hospital bed, raising/falling 55 of a third portion of a hospital bed, raising/falling 56 of a second portion (the whole) of a hospital bed. Alternatively, the controlled device 50 may be also one of volume adjustment 571 of a radio, channel adjustment 572 of a radio, channel adjustment 581 of a TV, volume adjustment 582 of a TV, temperature adjustment 591 or humidity adjustment 592.

Furthermore, the brain wave measurement device 30 is configured for measuring a brain wave signal induced by the user when gazing at the optical flash generating device 20, and transmitting the brain wave signal to the programmable chip 40. The brain wave signal measurement device 30 includes a brain measurement device 31, a biomedical signal instrument amplifier 32 and an analog-to-digital converter 33. The brain measurement device 31 may be one of 10-20 type systems designed by the International Brain Wave Association. The brain measurement device 31 employs a signal input electrode attached on a brain visual area 72, a reference electrode attached on a postauricular mastoid 73, and a grounding electrode attached on a forehead 71 to measure the brain wave signal. The measured brain wave signal is amplified by the biomedical signal instrument amplifier 32, and then converted from an analog signal to a digital signal by the analog-to-digital converter 33.

The programmable chip 40 may be one of a group consisting of a field programmable gate array, a microcomputer unit and a digital signal processor. The programmable chip 40 is configured for analyzing the relation between the brain wave signal and the optical flash generating device 20, and determining a selected option of the optical display region 21 gazed by the user. The controlled device 50 receives a signal sent out from the programmable chip 40, according to the selected option of the optical display region 21 gazed by the user, such that the state of the controlled device 50 can be controlled.

Referring to FIG. 2, the optical flash generating device 20 of the present invention is shown. Then, at least one optical display region 21 may respectively correspond to raising/falling 54 of the first portion of the hospital bed, raising/falling 55 of the third portion of the hospital bed, raising/falling 56 of the second portion (the whole) of the hospital bed, the volume adjustment 571 of the radio, the channel adjustment 572 of the radio, the channel adjustment 581 of the TV, the volume adjustment 582 of the TV, the temperature adjustment 591 and the humidity adjustment 592.

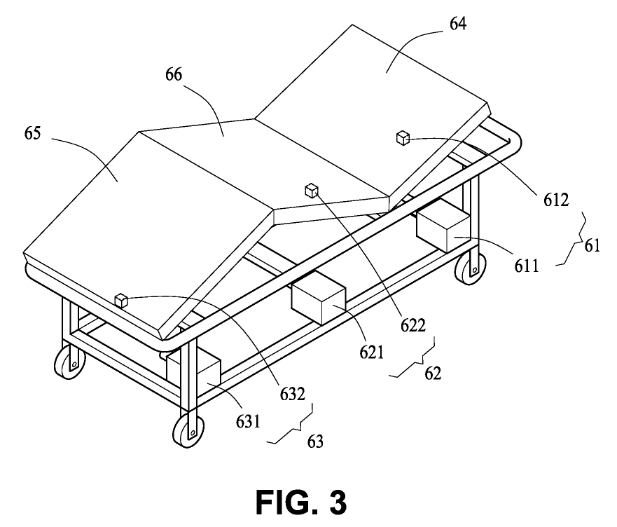

Referring to FIG. 3, the controlled device 50 of an exemplary embodiment of the present invention is shown. If the controlled device 50 is a hospital bed, a first lifter 61, a second lifter 62 and a third lifter 63 are arranged in the first portion 64, the second portion 66 and the third portion 65 of the hospital bed, respectively. The first lifter 61 includes a first motor 611 and a first sensor 612. The first motor 611 is configured for receiving the signal sent out from the programmable chip 40 and corresponding to the selected option (such as, raising/falling 54 of the first portion of the hospital bed as shown in FIG. 2) of the optical display region 21 gazed by the user to drive the first lifter 61, such the first portion 64 is raised/fallen with the first lifter 61.

The second lifter 62 includes a second motor 621 and a second sensor 622. The second motor 621 is configured for receiving the signal sent out from the programmable chip 40 and corresponding to the selected option (such as, raising/falling 56 of the second portion of the hospital bed as shown in FIG. 2) of the optical display region 21 gazed by the user to drive the second lifter 62, such the second portion 66 is raised/fallen with the second lifter 62.

The third lifter 63 includes a third motor 631 and a third sensor 632. The third motor 631 is configured for receiving the signal sent out from the programmable chip 40 and corresponding to the selected option (such as, raising/falling 55 of the third portion of the hospital bed as shown in FIG. 2) of the optical display region 21 gazed by the user to drive the third lifter 63, such the third portion 65 is raised/fallen with the third lifter 63.

Furthermore, the first sensor 612, the second sensor 622 and the third sensor 632 are configured for sensing the first portion 64, the second portion 66 and the third portion 65 respectively, generating respectively sensing signals when the first portion 64, the second portion 66 and the third portion 65 reach the top end or the bottom end thereof, and transmitting the sensing signals to the programmable chip 40 (as shown in FIG. 1). Thus the programmable chip 40 stop driving the corresponding first motor 611, the second motor 621 or the third motor 631 for protecting the user and the hospital bed.

From the above, the nursing system 10 of the visual evoked brain wave control device of the present invention is not controlled by the periphery neuron and muscle, and is controlled only by the brain wave of the user, such that the state of the hospital bed or the periphery devices are controlled. Thus, even if the user is paralyzed on the hospital beds and only has brains operating (such as, quadriplegic patient, amyotrophic lateral sclerosis patient, or cerebellar atrophy patient, etc.), the user also can use the present nursing system 10 of the visual evoked brain wave control device to control the state of the hospital bed or the periphery devices. The present nursing system 10 is novel and practicable.

The above description is given by way of example, and not limitation. Given the above disclosure, one skilled in the art could devise variations that are within the scope and spirit of the invention disclosed herein, including configurations ways of the recessed portions and materials and/or designs of the attaching structures. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A nursing system of a visual evoked brain wave control device, the nursing system being controlled by brain wave signals, and the nursing system comprising:
an optical flash generating device facing toward a user and spaced a predetermined distance from eyes of a user such that the user directly views at least one optical display region of the optical flash generating device, the optical flash generating device being configured for receiving an outer signal to drive and flash the at least one optical display region;
a brain wave measurement device configured for measuring a brain wave signal induced by the user when gazing at one optical display region, the brain wave measurement device including a brain measurement device, a biomedical signal instrument amplifier and an analog-to-digital converter arranged therein, the brain measurement device being configured for measuring the brain wave signal, the biomedical signal instrument amplifier being configured for amplifying the brain wave measured signal by the brain measurement device, and the analog-to-digital converter being configured for converting the amplified brain wave signal to a digital signal;
a programmable chip configured for receiving the brain wave sent out from the brain wave measurement device, analyzing a relation between the brain wave and the optical flash generating device, and determining a selected option of the optical display region gazed by the user; and
at least one controlled device configured for receiving a signal sent out from the programmable chip and corresponding to the selected option of the optical display region gazed by the user to control a state thereof.

2. The nursing system as claimed in claim 1, wherein the at least one optical display region corresponds to the at least one controlled device.

3. The nursing system as claimed in claim 2, wherein the brain measurement device is one of 10-20 type systems designed by the International Brain Wave Association, the brain measurement device includes a signal input electrode attached on a brain visual area, a reference electrode attached on a postauricular mastoid, and a grounding electrode attached on a forehead to measure at least one brain wave signal.

4. The nursing system as claimed in claim 2, wherein the programmable chip is one of a group consisting of a field programmable gate array, a microcomputer unit and a digital processor.

5. The nursing system as claimed in claim 2, wherein the controlled device is one of a group consisting of raising/falling of a first portion of a hospital bed, raising/falling of a third portion of a hospital bed, raising/falling of a second portion of a hospital bed, volume adjustment of a radio, channel adjustment of a radio, channel adjustment of a TV, volume adjustment of a TV, temperature adjustment and humidity adjustment.

6. The nursing system as claimed in claim 5, wherein a first lifter, a second lifter and a third lifter are arranged in the first portion of the hospital bed, the second portion and the third portion thereof respectively, the first lifter includes a first motor configured for receiving the signal sent out from the programmable chip and corresponding to the selected option of the optical display region gazed by the user, and driving the first lifter to raising/falling the first portion with the first lifter; the second lifter includes a second motor configured for receiving the signal sent out from the programmable chip and corresponding to the selected option of the optical display region gazed by the user, and driving the second lifter to raising/falling the second portion with the second lifter; and the third lifter includes a third motor configured for receiving the signal sent out from the programmable chip and corresponding to the selected option of the optical display region gazed by the user, and driving the third lifter to raising/falling the third portion with the third lifter.

7. The nursing system as claimed in claim 6, wherein the first lifter, the second lifter and the third lifter further includes respectively a first sensor, a second sensor and a third sensor, the first sensor, the second sensor, and the third sensor are respectively configured for sensing the first portion, the second portion and the third portion, generating sensing signals when they reach a top end or a bottom end thereof, and transmitting the sensing signals to the programmable chip such that the programmable chip stops driving the first motor, the second motor or the third motor.
